(19) United States
(12) Patent Application Publication
(10) Pub. No.: US 2013/0115629 A1
(43) Pub. Date: May 9, 2013

(54) METHODS OF BREAST CANCER DETECTION

(71) Applicant: Atossa Genetics, Inc., Seattle, WA (US)

(72) Inventors: Steven C. Quay, Seattle, WA (US); Shu-Chih Chen, Seattle, WA (US)

(73) Assignee: Atossa Genetics, Inc., Seattle, WA (US)

(21) Appl. No.: 13/659,831

(22) Filed: Oct. 24, 2012

Related U.S. Application Data
(60) Provisional application No. 61/550,865, filed on Oct. 24, 2011.

(51) Int. Cl. G01N 33/574

(52) U.S. Cl. CPC G01N 33/57415
USPC 435/7.23; 435/287.2

(57) ABSTRACT

Methods of diagnosing breast cancers are provided. The methods include analyzing the expression patterns of biomarkers in a plurality of cells to distinguish between invasive and non-invasive cancers, usual and atypical hyperplasias, and basal-like breast cancers. Breast cancers are diagnosed based upon different combinations of the biomarkers found in nipple aspirate fluid.

### CROSS-REFERENCE

This application claims the benefit of U.S. Provisional Application No. 61/550,865, filed Oct. 24, 2011, which application is incorporated herein by reference.

### BACKGROUND OF THE INVENTION

Breast cancer is cancer originating from breast tissue, most commonly from the inner lining of milk ducts or the lobules that supply the ducts with milk. Cancers originating from ducts are known as ductal carcinomas; those originating from lobules are known as lobular carcinomas.

### SUMMARY OF THE INVENTION

Disclosed herein, in certain embodiments, are methods of classifying a breast cancer as basal-like, comprising: (a) contacting a plurality of cells derived from a nipple aspirate fluid (NAF) sample with primary antibodies that bind to CK5, CK14, CK7, CK18, and p63; and (b) classifying the cancer as basal-like if the CK5, CK14, and optionally anti-p63 primary antibodies bind to the plurality of cells; wherein the plurality of cells derived from a nipple aspirate fluid (NAF) sample are not a tissue. In some embodiments, the methods further comprise contacting the plurality of cells with a first population of secondary antibodies that bind to CK5, CK14, and p63, and a second population of secondary antibodies that bind to CK7 and CK18. In some embodiments, the first population of secondary antibodies comprises horseradish peroxidase (HRP), and the second population of secondary antibodies comprises alkaline phosphatase (AP). In some embodiments, the methods further comprise contacting the plurality of cells with diaminobenzidine (DAB), and Fast Red (FR). In some embodiments, the methods further comprise counterstaining the plurality of cells with hematoxylin. In some embodiments, the plurality of cells are visualized with a light microscope. In some embodiments, the plurality of cells are visualized with an automated system. In some embodiments, the methods further comprise contacting the plurality of cells with a peroxide block before contact with the secondary antibodies. In some embodiments, the methods further comprise collecting the plurality of cells derived from NAF on an absorbent paper. In some embodiments, the methods further comprise collecting microscopthe plurality of cells derived from NAF on an absorbent paper comprising microcellulose, mixed cellulose ester, or nitrocellulose. In some embodiments, the methods further comprise washing the absorbent paper and collecting the effluent. In some embodiments, the plurality of cells is at least two cells. In some embodiments, the plurality of cells is more than two cells. In some embodiments, the NAF sample is obtained from a classical non-secretor or a classical secretor of nipple aspirate fluid. In some embodiments, the plurality of cells are triple negative. In some embodiments, contacting the plurality of cells derived from a nipple aspirate fluid (NAF) sample with the primary antibodies is automated. In some embodiments, contacting the plurality of cells derived from a nipple aspirate fluid (NAF) sample with the secondary antibodies is automated. In some embodiments, wherein contacting the plurality of cells derived from a nipple aspirate fluid (NAF) sample with diaminobenzidine (DAB), and Fast Red (FR) is automated.

Disclosed herein, in certain embodiments, are methods of classifying a breast cancer as luminal, comprising: (a) contacting a plurality of cells derived from a nipple aspirate fluid (NAF) sample with primary antibodies that bind to CK5, CK14, CK7, CK18, and p63; and (b) classifying the cancer as luminal if (i) the anti-CK7 and anti-CK1 8 primary antibodies bind to the plurality of cells, and (ii) the anti-CK5, anti-CK14, and anti-p63 primary antibodies do not bind to the plurality of cells; wherein the plurality of cells derived from a nipple aspirate fluid (NAF) sample are not a tissue. In some embodiments, the methods further comprise contacting the plurality of cells with a first population of secondary antibodies that bind to CK5, CK14, and p63, and a second population of secondary antibodies that bind to CK7 and CK18. In some embodiments, the first population of secondary antibodies comprises horseradish peroxidase (HRP), and the second population of secondary antibodies comprises alkaline phosphatase (AP). In some embodiments, the methods further comprise contacting the plurality of cells with diaminobenzidine (DAB), and Fast Red (FR). In some embodiments, the methods further comprise counterstaining the plurality of cells with hematoxylin. In some embodiments, the plurality of cells are visualized with a light microscope. In some embodiments, the plurality of cells are visualized with an automated system. In some embodiments, the methods further comprise contacting the plurality of cells with a peroxide block before contact with the secondary antibodies. In some embodiments, the methods further comprise collecting the plurality of cells derived from NAF on an absorbent paper. In some embodiments, the methods further comprise collecting the plurality of cells derived from NAF on an absorbent paper comprising microcellulose, mixed cellulose ester, or nitrocellulose. In some embodiments, the methods further comprise washing the absorbent paper and collecting the effluent. In some embodiments, the plurality of cells is at least two cells. In some embodiments, the plurality of cells is more than two cells. In some embodiments, the NAF sample is obtained from a classical non-secretor or a classical secretor of nipple aspirate fluid. In some embodiments, the plurality of cells are triple negative. In some embodiments, contacting the plurality of cells derived from a nipple aspirate fluid (NAF) sample with the primary antibodies is automated. In some embodiments, contacting the plurality of cells derived from a nipple aspirate fluid (NAF) sample with the secondary antibodies is automated. In some embodiments, wherein contacting the plurality of cells derived from a nipple aspirate fluid (NAF) sample with diaminobenzidine (DAB), and Fast Red (FR) is automated.

Disclosed herein, in certain embodiments, are methods of classifying a hyperplasia usual ductal hyoperplasia, comprising: (a) contacting a plurality of cells derived from a nipple aspirate fluid (NAF) sample with primary antibodies that bind to CK5, CK14, CK7, CK18, and p63; and (b) classifying the hyperplasia as an usual ductal hyperplasia if the CK5, CK14, CK7, CK18, and p63 primary antibodies bind to the plurality of cells; wherein the plurality of cells derived from a nipple aspirate fluid (NAF) sample are not a tissue. In some embodiments, the methods further comprise contacting the plurality of cells with a first population of secondary antibodies that bind to CK5, CK14, and p63, and a second population of secondary antibodies that bind to CK7 and CK18. In some embodiments, the first population of secondary antibodies comprises horseradish peroxidase (HRP), and the second population of secondary antibodies comprises alkaline phosphatase (AP). In some embodiments, the methods further comprise contacting the plurality of cells with diaminobenzidine (DAB), and Fast Red (FR). In some embodiments, the methods further comprise counterstaining the plurality of cells with hematoxylin. In some embodiments, the plurality of cells are visualized with a light microscope. In some embodiments, the plurality of cells are visualized with an automated system. In some embodiments, the methods further comprise contacting the plurality of cells with a peroxide block before contact with the secondary antibodies. In some embodiments, the methods further comprise collecting the plurality of cells derived from NAF on an absorbent paper. In some embodiments, the methods further comprise collecting the plurality of cells derived from NAF on an absorbent paper comprising microcellulose, mixed cellulose ester, or nitrocellulose. In some embodiments, the methods further comprise washing the absorbent paper and collecting the effluent. In some embodiments, the plurality of cells is at least two cells. In some embodiments, the plurality of cells is more than two cells. In some embodiments, the NAF sample is obtained from a classical non-secretor or a classical secretor of nipple aspirate fluid. In some embodiments, the plurality of cells are triple negative. In some embodiments, contacting the plurality of cells derived from a nipple aspirate fluid (NAF) sample with the primary antibodies is automated. In some embodiments, contacting the plurality of cells derived from a nipple aspirate fluid (NAF) sample with the secondary antibodies is automated. In some embodiments, wherein contacting the plurality of cells derived from a nipple aspirate fluid (NAF) sample with diaminobenzidine (DAB), and Fast Red (FR) is automated.

Disclosed herein, in certain embodiments, are methods of classifying a hyperplasia as atypical ductal hyoperplasia, comprising: (a) contacting a plurality of cells derived from a nipple aspirate fluid (NAF) sample with primary antibodies that bind to CK5, CK14, CK7, CK18, and p63; and (b) classifying the hyperplasia as atypical ductal hyperplasia if (i) the CK7 and CK18, and optionally the p63, primary antibodies bind to the plurality of cells, and (ii) the CK5 and CK14 primary antibodies do not bind to the plurality of cells; wherein the plurality of cells derived from a nipple aspirate fluid (NAF) sample are not a tissue. In some embodiments, the methods further comprise contacting the plurality of cells with a first population of secondary antibodies that bind to CK5, CK14, and p63, and a second population of secondary antibodies that bind to CK7 and CK18. In some embodiments, the first population of secondary antibodies comprises horseradish peroxidase (HRP), and the second population of secondary antibodies comprises alkaline phosphatase (AP). In some embodiments, the methods further comprise contacting the plurality of cells with diaminobenzidine (DAB), and Fast Red (FR). In some embodiments, the methods further comprise counterstaining the plurality of cells with hematoxylin. In some embodiments, the plurality of cells are visualized with a light microscope. In some embodiments, the plurality of cells are visualized with an automated system. In some embodiments, the methods further comprise contacting the plurality of cells with a peroxide block before contact with the secondary antibodies. In some embodiments, the methods further comprise collecting the plurality of cells derived from NAF on an absorbent paper. In some embodiments, the methods further comprise collecting the plurality of cells derived from NAF on an absorbent paper comprising microcellulose, mixed cellulose ester, or nitrocellulose. In some embodiments, the methods further comprise washing the absorbent paper and collecting the effluent. In some embodiments, the plurality of cells is at least two cells. In some embodiments, the plurality of cells is more than two cells. In some embodiments, the NAF sample is obtained from a classical non-secretor or a classical secretor of nipple aspirate fluid. In some embodiments, the plurality of cells are triple negative. In some embodiments, contacting the plurality of cells derived from a nipple aspirate fluid (NAF) sample with the primary antibodies is automated. In some embodiments, contacting the plurality of cells derived from a nipple aspirate fluid (NAF) sample with the secondary antibodies is automated. In some embodiments, wherein contacting the plurality of cells derived from a nipple aspirate fluid (NAF) sample with diaminobenzidine (DAB), and Fast Red (FR) is automated.

Disclosed herein, in certain embodiments, are methods of classifying a breast cancer as invasive, comprising: (a) contacting a plurality of cells derived from a nipple aspirate fluid (NAF) sample with primary antibodies that bind to CK5, CK14, CK7, CK18, and p63; and (b) classifying the cancer as invasive if the ratio of cells binding the CK5, CK14, and p63 primary antibodies to cells binding the CK7 and CK18 primary antibodies is less than or equal to an invasive control; wherein the plurality of cells derived from a nipple aspirate fluid (NAF) sample are not a tissue. In some embodiments, no cells bind CK5, CK14, and p63. In some embodiments, the methods further comprise contacting the plurality of cells with a first population of secondary antibodies that bind to CK5, CK14, and p63, and a second population of secondary antibodies that bind to CK7 and CK18. In some embodiments, the first population of secondary antibodies comprises horseradish peroxidase (HRP), and the second population of secondary antibodies comprises alkaline phosphatase (AP). In some embodiments, the methods further comprise contacting the plurality of cells with diaminobenzidine (DAB), and Fast Red (FR). In some embodiments, the methods further comprise counterstaining the plurality of cells with hematoxylin. In some embodiments, the plurality of cells are visualized with a light microscope. In some embodiments, the plurality of cells are visualized with an automated system. In some embodiments, the methods further comprise contacting the plurality of cells with a peroxide block before contact with the secondary antibodies. In some embodiments, the methods further comprise collecting the plurality of cells derived from NAF on an absorbent paper. In some embodiments, the methods further comprise collecting the plurality of cells derived from NAF on an absorbent paper comprising microcellulose, mixed cellulose ester, or nitrocellulose. In some embodiments, the methods further comprise washing the absorbent paper and collecting the effluent. In some embodiments, the plurality of cells is at least two cells. In some embodiments, the plurality of cells is more than two cells. In some embodiments, the NAF sample is obtained from a classical non-secretor or a classical secretor of nipple aspirate fluid. In some embodiments, the plurality of cells are triple negative. In some embodiments, contacting the plurality of cells derived from a nipple aspirate fluid (NAF) sample with the primary antibodies is automated. In some embodiments, contacting the plurality of cells derived from a nipple aspirate fluid (NAF) sample with the secondary antibodies is automated. In some embodiments, wherein contacting the plurality of cells derived from a nipple aspirate fluid (NAF) sample with diaminobenzidine (DAB), and Fast Red (FR) is automated.

Disclosed herein, in certain embodiments, are methods of classifying a breast cancer as non-invasive, comprising: (a) contacting a plurality of cells derived from a nipple aspirate fluid (NAF) sample with primary antibodies that bind to CK5, CK14, CK7, CK18, and p63; and (b) classifying the cancer as non-invasive if the ratio of cells binding the CK5, CK14, and p63 primary antibodies to cells binding the CK7 and CK18 primary antibodies is greater than or equal to a non-invasive control; wherein the plurality of cells derived from a nipple aspirate fluid (NAF) sample are not a tissue. In some embodiments, the methods further comprise contacting the plurality of cells with a first population of secondary antibodies that bind to CK5, CK14, and p63, and a second population of secondary antibodies that bind to CK7 and CK18. In some embodiments, the first population of secondary antibodies comprises horseradish peroxidase (HRP), and the second population of secondary antibodies comprises alkaline phosphatase (AP). In some embodiments, the methods further comprise contacting the plurality of cells with diaminobenzidine (DAB), and Fast Red (FR). In some embodiments, the methods further comprise counterstaining the plurality of cells with hematoxylin. In some embodiments, the plurality of cells are visualized with a light microscope. In some embodiments, the plurality of cells are visualized with an automated system. In some embodiments, the methods further comprise contacting the plurality of cells with a peroxide block before contact with the secondary antibodies. In some embodiments, the methods further comprise collecting the plurality of cells derived from NAF on an absorbent paper. In some embodiments, the methods further comprise collecting the plurality of cells derived from NAF on an absorbent paper comprising microcellulose, mixed cellulose ester, or nitrocellulose. In some embodiments, the methods further comprise washing the absorbent paper and collecting the effluent. In some embodiments, the plurality of cells is at least two cells. In some embodiments, the plurality of cells is more than two cells. In some embodiments, the NAF sample is obtained from a classical non-secretor or a classical secretor of nipple aspirate fluid. In some embodiments, the plurality of cells are triple negative. In some embodiments, contacting the plurality of cells derived from a nipple aspirate fluid (NAF) sample with the primary antibodies is automated. In some embodiments, contacting the plurality of cells derived from a nipple aspirate fluid (NAF) sample with the secondary antibodies is automated. In some embodiments, wherein contacting the plurality of cells derived from a nipple aspirate fluid (NAF) sample with diaminobenzidine (DAB), and Fast Red (FR) is automated.

Provided herein is a system for classifying a breast cancer, comprising: an absorbent paper comprising microcellulose, mixed cellulose ester, or nitrocellulose for absorbing a nipple aspirate fluid sample, wherein the absorbent paper is sized to cover a nipple and is from about 1.0 to about 3.0 inches in diameter and from about 0.01 to about 0.1 inches thick; antibodies that bind to an antigens on a cell in the nipple aspirate fluid sample, wherein the antigens are selected from: CK5, CK14, CK7, CK18, p63, CK7 and CK18; and a light microscope or an automated system for visualizing the antibodies bound to a cell in the nipple aspirate fluid sample. The system may further comprise one or more means for visualizing the antibodies bound to the cell in the nipple aspirate fluid sample. In one embodiment, the means for visualizing the antibodies bound to the cell in the nipple aspirate fluid sample is one or more stains. Stains include, but are not limited to, horseradish peroxidase, alkaline phosphatase, diaminobenzidine, Fast Red, hematoxylin, eosin or a combination thereof. The system may also further comprise a wash for eluting a cell in the nipple aspirate fluid sample from the absorbent paper. The system may also further comprise an optionally networked computer processing device configured to perform executable instructions; and a computer program, the computer program comprising a software module executed by the computer processing device to apply a model or algorithm for analyzing said cells. In one embodiment, the computer program further comprises a software module executed by the computer processing device to designate a treatment regimen for the individual. In another embodiment, the computer program further comprises a software module executed by the computer processing device to store photomicrograms in a database of photomicrograms. In another embodiment, the computer program further comprises a software module executed by the computer processing device to store analysis in a database of analyses. In another embodiment, the computer program further comprises a software module executed by the computer processing device to compare a cell in a nipple aspirate fluid sample to a standard. In another embodiment, the computer program further comprises a software module executed by the computer processing device to transmit an analysis to a health care provider or the individual. In another embodiment, the computer program further comprises a software module executed by the computer processing device to transmit a diagnosis to a health care provider or the individual. In another embodiment, the computer program further comprises a software module executed by the computer processing device to generate a report comprising the analysis. In another embodiment, the absorbent paper is a device as illustrated in FIG. 1.

Provided herein is a non-transitory computer-readable storage media encoded with a computer program including instructions executable by a computer processing device to create an application, the application comprising: (a) a software module configured to apply a model or algorithm for analyzing a cell of a nipple aspirate fluid sample absorbed onto an absorbent paper that is sized to cover a nipple and is from about 1.0 to about 3.0 inches in diameter and from about 0.01 to about 0.1 inches thick, and wherein the cell further comprises an antibody that binds to: CK5, CK14, CK7, CK18, p63, CK7 and CK18; and (b) a software module configured to designate a treatment regimen for the individual. In one embodiment, the model or algorithm compares the cell to a standard. In one embodiment, the application further comprises a database, in a computer memory, of photomicrographs. In one embodiment, the application further comprises a database, in a computer memory, of analyses. In one embodiment, the application further comprises a software module configured to generate a report comprising the analysis. In one embodiment, the absorbent paper is a device as illustrated in FIG. 1.

Provided herein is a method of classifying a breast cancer, comprising: contacting a cell of a nipple aspirate fluid sample absorbed onto an absorbent paper with antibodies that bind to CK5, CK14, CK7, CK18, and p63, wherein the absorbent paper is sized to cover a nipple and is from about 1.0 to about 3.0 inches in diameter and from about 0.01 to about 0.1 inches thick; detecting binding of one or more of the antibodies to said cell; and classifying the cancer based upon the binding pattern of the primary antibodies; wherein the cell derived from the nipple aspirate fluid (NAF) sample is not a tissue. In one embodiment, detecting binding of the one or more antibodies further comprises staining the cells with a stain selected from among horseradish peroxidase, alkaline phosphatase, diaminobenzidine, Fast Red, hematoxylin, eosin and a combination thereof. In one embodiment, the method further comprises washing the absorbent paper and collecting the effluent. An absorbent paper may be, for example, microcellulose, mixed cellulose ester, or nitrocellulose. In one embodiment, the absorbent paper is a device as illustrated in FIG. 1. The method may further comprise classifying the breast cancer as basal-like if an anti-CK5 antibody, an anti-CK14 antibody, and optionally an anti-p63, primary antibody binds to the cell. The method may further comprise classifying the breast cancer as luminal if (i) an anti-CK7 antibody and an anti-CK18 primary antibody bind to the cell, and (ii) an anti-CK5 antibody, and anti-CK14 antibody, and an anti-p63 antibody do not bind to the cell. The method may further comprise classifying the breast cancer as usual ductal hyperplasia if an anti-CK5 antibody, an anti-CK14 antibody, an anti-CK7 antibody, an anti-CK18 antibody, and an anti-p63 primary antibody bind to the cell. The method may further comprise classifying the cancer as atypical ductal hyperplasia if (i) the anti-CK7 antibody and anti-CK18 antibody, and optionally the anti-p63 antibody, bind to the cell, and (ii) the anti-CK5 antibody and anti-CK14 antibody do not bind to the cell. The method may further comprise classifying the cancer as invasive if (i) the sample comprises more than one cell and (ii) the ratio of cells binding the anti-CK5 antibody, anti-CK14 antibody, and anti-p63 antibody to cells binding the anti-CK7 antibody and anti-CK18 antibody is less than or equal to an invasive control. The method may further comprise classifying the cancer as non-invasive if: the sample comprises more than one cell, and the ratio of cells binding the anti-CK5 antibody, anti-CK14 antibody, and anti-p63 antibody to cells binding the anti-CK7 antibody and anti-CK18 antibody is greater than or equal to a non-invasive control. The NAF sample to be used in such methods may be obtained from a classical non-secretor or a classical secretor of nipple aspirate fluid.

Disclosed herein, in certain embodiments, is a composition comprising (a) at least one cell derived from nipple aspirate fluid absorbed onto an absorbent paper comprising microcellulose, mixed cellulose ester, or nitrocellulose for absorbing a nipple aspirate fluid sample, wherein the absorbent paper is sized to cover a nipple; and (b) antibodies that bind to an antigens on a cell in the nipple aspirate fluid sample, wherein the antigens are selected from: CK5, CK14, CK7, CK18, p63, CK7 and CK18.

Disclosed herein, in certain embodiments, is a system for classifying a breast cancer, comprising: (a) an absorbent paper comprising microcellulose, mixed cellulose ester, or nitrocellulose for absorbing a nipple aspirate fluid sample, wherein the absorbent paper is sized to cover a nipple; and (b) antibodies that bind to an antigens on a cell in the nipple aspirate fluid sample, wherein the antigens are selected from: CK5, CK14, CK7, CK18, p63, CK7 and CK18. In some embodiments, the system further comprises a light microscope or an automated system for visualizing the antibodies bound to a cell in the nipple aspirate fluid sample. In some embodiments, the absorbent paper is from about 1.0 to about 3.0 inches in diameter and from about 0.01 to about 0.1 inches thick. In some embodiments, the system further comprises means for visualizing the antibodies bound to the cell in the nipple aspirate fluid sample. In some embodiments, the means for visualizing the antibodies bound to the cell in the nipple aspirate fluid sample is one or more stains. In some embodiments, the stain is selected from horseradish peroxidase, alkaline phosphatase, diaminobenzidine, Fast Red, hematoxylin, eosin or a combination thereof. In some embodiments, the system further comprises a wash for eluting a cell in the nipple aspirate fluid sample from the absorbent paper. In some embodiments, the system further comprises: an optionally networked computer processing device configured to perform executable instructions; and a computer program, the computer program comprising a software module executed by the computer processing device to apply a model or algorithm for analyzing said cells. In some embodiments, the computer program further comprises a software module executed by the computer processing device to designate a treatment regimen for the individual. In some embodiments, the computer program further comprises a software module executed by the computer processing device to store photomicrograms in a database of photomicrograms. In some embodiments, the computer program further comprises a software module executed by the computer processing device to store analysis in a database of analyses. In some embodiments, the computer program further comprises a software module executed by the computer processing device to compare a cell in a nipple aspirate fluid sample to a standard. In some embodiments, the computer program further comprises a software module executed by the computer processing device to transmit an analysis to a health care provider or the individual. In some embodiments, the computer program further comprises a software module executed by the computer processing device to transmit a diagnosis to a health care provider or the individual. In some embodiments, the computer program further comprises a software module executed by the computer processing device to generate a report comprising the analysis. In some embodiments, the absorbent paper is a device as illustrated in FIG. 1.

### BRIEF DESCRIPTION OF THE DRAWINGS

The features of the disclosure are set forth with particularity in the appended claims. A better understanding of the features and advantages of the present compositions, kits and methods will be obtained by reference to the following detailed description that sets forth illustrative embodiments, in which the principles of the disclosure embodiments are utilized, and the accompanying drawings of which:

FIGS. 1A-B illustrate a 2-D image of a representative absorbent paper or membrane described herein. Angular dimensions are provided in inches ±1° and in degrees. FIG. 1A illustrates a top view of the paper or membrane. FIG. 1B illustrates a side angle production.

### DETAILED DESCRIPTION OF THE INVENTION

Although there are currently several methods for molecular subtyping breast cancer, more sensitive and reliable methods are needed. Preferably, these methods are non-invasive, do not require tissue samples, and may be used with individuals classified as secretors or non-secretors of nipple aspirate fluid (NAF).

The methods of the embodiments provided herein may be conducted with an appropriate breast pump device which may be used for sample collection such as, for example a device described in U.S. Pat. No. 5,798,266; U.S. Pat. No. 6,689,073; and U.S. Pat. No. 6,887,210, each of which is incorporated herein by reference. In one embodiment, the device is a MASCT™ device.

In some embodiments, methods disclosed herein require less than 10 cells, less than 9 cells, less than 8 cells, less than 7 cells, less than 5 cells, less than 4 cells, less than 3 cells, less than 2 cells. More preferably, these methods require two cells.

Disclosed herein, in certain embodiments, are methods of classifying a breast cancer as basal-like, comprising: (a) contacting a plurality of cells derived from a nipple aspirate fluid (NAF) sample with primary antibodies that bind to CK5, CK14, CK7, CK18, and p63; and (b) classifying the cancer as basal-like if the CK5, CK14, and optionally anti-p63 primary antibodies bind to the plurality of cells; wherein the plurality of cells derived from a nipple aspirate fluid (NAF) sample are not a tissue.

Disclosed herein, in certain embodiments, are methods of classifying a breast cancer as luminal, comprising: (a) contacting a plurality of cells derived from a nipple aspirate fluid (NAF) sample with primary antibodies that bind to CK5, CK14, CK7, CK18, and p63; and (b) classifying the cancer as luminal if (i) the anti-CK7 and anti-CK18 primary antibodies bind to the plurality of cells, and (ii) the anti-CK5, anti-CK14, and anti-p63 primary antibodies do not bind to the plurality of cells; wherein the plurality of cells derived from a nipple aspirate fluid (NAF) sample are not a tissue.

Disclosed herein, in certain embodiments, are methods of classifying a hyperplasia usual ductal hyoperplasia, comprising: (a) contacting a plurality of cells derived from a nipple aspirate fluid (NAF) sample with primary antibodies that bind to CK5, CK14, CK7, CK18, and p63; and (b) classifying the hyperplasia as an usual ductal hyperplasia if the CK5, CK14, CK7, CK18, and p63 primary antibodies bind to the plurality of cells; wherein the plurality of cells derived from a nipple aspirate fluid (NAF) sample are not a tissue.

Disclosed herein, in certain embodiments, are methods of classifying a hyperplasia as atypical ductal hyoperplasia, comprising: (a) contacting a plurality of cells derived from a nipple aspirate fluid (NAF) sample with primary antibodies that bind to CK5, CK14, CK7, CK18, and p63; and (b) classifying the hyperplasia as atypical ductal hyperplasia if (i) the CK7 and CK18, and optionally the p63, primary antibodies bind to the plurality of cells, and (ii) the CK5 and CK14 primary antibodies do not bind to the plurality of cells; wherein the plurality of cells derived from a nipple aspirate fluid (NAF) sample are not a tissue.

Disclosed herein, in certain embodiments, are methods of classifying a breast cancer as invasive, comprising: (a) contacting a plurality of cells derived from a nipple aspirate fluid (NAF) sample with primary antibodies that bind to CK5, CK14, CK7, CK18, and p63; and (b) classifying the cancer as invasive if the ratio of cells binding the CK5, CK14, and p63 primary antibodies to cells binding the CK7 and CK18 primary antibodies is less than or equal to an invasive control; wherein the plurality of cells derived from a nipple aspirate fluid (NAF) sample are not a tissue. In some embodiments, no cells bind CK5, CK14, and p63.

Disclosed herein, in certain embodiments, are methods of classifying a breast cancer as non-invasive, comprising: (a) contacting a plurality of cells derived from a nipple aspirate fluid (NAF) sample with primary antibodies that bind to CK5, CK14, CK7, CK18, and p63; and (b) classifying the cancer as non-invasive if the ratio of cells binding the CK5, CK14, and p63 primary antibodies to cells binding the CK7 and CK18 primary antibodies is greater than or equal to a non-invasive control; wherein the plurality of cells derived from a nipple aspirate fluid (NAF) sample are not a tissue.

### Definitions

As used herein, the term “treatment”, “treat”, or “treating” in some embodiments includes achieving a therapeutic benefit. Therapeutic benefit is meant to include eradication or amelioration of the underlying disorder or condition being treated. For example, therapeutic benefit includes alleviation or partial and/or complete halting of a sleep-related breathing disorder. Also, a therapeutic benefit is achieved with the eradication or amelioration of one or more of the physiological or psychological symptoms associated with the underlying condition such that an improvement is observed in the patient, notwithstanding the fact that the patient is still affected by the condition. For example, in an individual suffering from a sleep-related breathing disorder, therapeutic benefit includes alleviation or partial and/or complete halting of sleep fragmentation, or reduction in frequency of arousals or awakenings or reduction in incidence of awakenings. In some embodiments, “treatment” provides prophylactic benefit including prevention of a condition, retarding the progress of a condition, or decreasing the likelihood of occurrence of a condition (e.g., prevention of the sleep-related breathing disorder). As used herein, “treat”, “treating” or “treatment” includes prophylaxis.

As used herein, “administer” means to provide a treatment, for example to prescribe a treatment, apply a treatment, or distribute a treatment. In some instances, to administer means a medical professional prescribes a treatment which a patient applies (e.g., the patient applies a CPAP device, consumes a medication, or injects a medication). Administration of a medical treatment does not require the immediate or constant supervision of a medical professional.

### Breast Disorders

The normal breast consists of ducts and lobules with a dual-layered architecture. Luminal secretory cells surround a hollow lumen, and in turn are surrounded by a layer of myoepithelial cells that lie in direct contact with the basement membrane.

### Breast Hyperplasia

Hyperplasia (also known as epithelial hyperplasia or proliferative breast disease) is an overgrowth of the cells that line either the ducts or the lobules. When hyperplasia is in the duct, it is called ductal hyperplasia or duct epithelial hyperplasia. When it affects the lobule, it is referred to as lobular hyperplasia.

Hyperplasia is usually diagnosed with a core needle biopsy or surgical biopsy. Based on how the cells look under the microscope, hyperplasia may be grouped as:

Mild hyperplasia: This does not increase the risk for breast cancer

Hyperplasia of the usual type (without atypia), also known as usual hyperplasia: The risk of breast cancer is about 1½ to 2 times that of a woman with no breast abnormalities.

Atypical hyperplasia (either atypical ductal hyperplasia [ADH] or atypical lobular hyperplasia [ALH]): The risk of breast cancer is about 4 to 5 times higher than that of a woman with no breast abnormalities.

### Breast Cancers

Breast cancer usually begins either in the cells of the lobules or the ducts. A breast cancer may be a “mixed tumor,” meaning that it contains a mixture of cancerous ductal cells and lobular cells. In such cases, the cancer is treated as a ductal carcinoma. If there is more than one tumor in the breast, the breast cancer is described as either multifocal or multicentric. In multifocal breast cancer, all of the tumors arise from the original tumor, and they are usually in the same section of the breast. If the cancer is multicentric, it means that all of the tumors formed separately, and they are often in different areas of the breast.

Invasive vs. Non-Invasive

Non-invasive cancers stay within the ducts or lobules in the breast. They do not grow into or invade normal tissues within or beyond the breast. Non-invasive cancers are sometimes called carcinoma in situ (“in the same place”) or pre-cancers. Invasive cancers grow into normal, healthy tissues. Most breast cancers are invasive. Whether the cancer is non-invasive or invasive will affect treatment choices and responses thereto.

A breast cancer may be both invasive and non-invasive. This means that part of the cancer has grown into normal tissue and part of the cancer has stayed inside the milk ducts or milk lobules. In such cases, these cancers would be treated as an invasive.

In most cases, a breast cancer is classified as one of the following:

DCIS (Ductal Carcinoma In Situ): DCIS is a non-invasive cancer that stays inside the milk duct.

MIC (Microinvasive breast carcinoma, MICB, and DCISM): MIC is a subtype of DCIS. It has a size that is less than 1.0 mm and about 10% or less of MIC cells have left the duct tissue (the original tumor site).

LCIS (Lobular Carcinoma In Situ): LCIS is an overgrowth of cells that stay inside the lobule. It indicates an increased risk for developing an invasive cancer.

IDC (Invasive Ductal Carcinoma): The most common type of breast cancer, invasive ductal carcinoma begins in the milk duct but has grown into the surrounding normal tissue inside the breast.

ILC (Invasive Lobular Carcinoma): ILC starts inside the lobule but grows into the surrounding normal tissue inside the breast.

Prevalence and Tumor Characteristics of Different Types
and Special Forms of Invasive Breast Cancer

| | Proportion | | |
|---|---|---|---|
| | of all | | |
| Types of | invasive | | |
| invasive | breast | Tumor | |
| breast cancer | cancers | characteristics | Prognosis |
| Invasive ductal | 50-75% | Hard | Prognosis varies with stage |
| carcinoma | | tumor texture | and grade of tumor |
| (IDC) | | Tumor is | |
| | | irregular, star- | |
| | | shaped | |
| | | Cell features | |
| | | vary | |
| | | DCIS often | |
| | | present | |
| Invasive | 10-15% | Normal, | Prognosis varies with stage |
| lobular | | slightly firm or | and grade of tumor |
| carcinoma | | hard | For any given stage or |
| (ILC) | | tumor texture | grade, prognosis is similar |
| | | Cells appear in | to that of IDC |
| | | single file order | Pattern of metastases is |
| | | Tumors | slightly different from IDC |
| | | are most | (more likely to go to the |
| | | often ER- | gastrointestinal tract) |
| | | positive and | |
| | | HER2/neu- | |
| | | negative | |
| Medullary | 1-5% | Soft tumor | More common among |
| carcinoma | | Cells have a | younger women and |
| | | sheet-like | women with a BRCA1 |
| | | appearance | genetic mutation |
| | | Tumors are | At this time, it is not known |
| | | often ER- | whether prognosis is better |
| | | negative | than or similar to that for |
| | | | IDC and ILC |
| Mucinous | 1-5% | Soft tumor | More common among older |
| (colloid) | | Often no | women |
| carcinoma | | palpable tumor | Tends to have a |
| | | Cells are | good prognosis |
| | | surrounded by | Less common for cancer to |
| | | excess mucous | spread to lymph nodes |
| | | (mucin) | |
| | | Tumors | |
| | | are most | |
| | | often ER- | |
| | | positive and | |
| | | HER2/neu- | |
| | | negative | |
| Papillary | 1-5% | Soft tumor | More common among |
| carcinoma | | Cells appear as | postmenopausal women |
| | | fingerlike | Tends to have a good |
| | | branches | prognosis |
| Tubular | 1-5%* | Tumors are | Prognosis is usually better |
| carcinoma | | most often | than IDC (survival at 5 |
| | | small | years is 88%) |
| | | Often no | Rare for cancer to spread to |
| | | palpable tumor | lymph nodes or other parts |
| | | Cells form | of the body |
| | | tube-like | |
| | | structures | |
| | | Tumors | |
| | | are most | |
| | | often ER- | |
| | | positive and | |
| | | HER2/neu- | |
| | | negative | |

### Molecular Subtypes

Gene expression profiling classifies breast cancers into four major biologically distinct intrinsic subtypes: luminal A, luminal B, human epidermal growth factor receptor-2 (HER2) over-expressing, and basal-like/triple negative. These molecular subtypes have prognostic and predictive value. The prognosis and chemotherapy sensitivity of the different molecular subgroups are different.

### Luminal Cancers

Most breast cancers are luminal tumors. Luminal tumor cells look like the cells of breast cancers that start in the inner (luminal) cells lining the mammary ducts.

Luminal A breast cancers are ER+ and/or PR+, HER2−, low Ki67. About 42-59% of breast cancers are luminal A. Luminal A tumors tend to be of low or moderate tumor grade. Of the four subtypes, luminal A tumors tend to have the best prognosis, with fairly high survival rates and fairly low recurrence rates. Only about 15% of luminal A tumors have p53 mutations, a factor linked with a poorer prognosis.

Luminal B breast cancers are ER+ and/or PR+, HER2+ (or HER2− with high Ki67). About 6-17% of breast cancers are luminal B. Women with luminal B tumors are often diagnosed at a younger age than those with luminal A tumors. Compared to luminal A tumors, luminal B tumors also tend to have factors that lead to a poorer prognosis including: poorer tumor grade; larger tumor size; and p53 gene mutations. In general, women with luminal B tumors have fairly high survival rates, although not as high as those with luminal A tumors.

### Basal-Like

Approximately 14-20% of breast cancers are basal-like. Basal-like breast cancers differ to luminal cancers in being triple negative for the immunophenotypic markers ER−/PR−/HER2− but express CK5/6. Basal-like breast cancers show increased hypoxia and high tumor grade and have an aggressive phenotype characterized by high cell proliferation and poor clinical outcome. Most BRCA1 breast cancers and many BRCA2 breast cancers are both triple negative/basal-like. Triple negative/basal-like tumors are often aggressive and have a poorer prognosis compared to the estrogen receptor-positive subtypes (luminal A and luminal B tumors). Triple negative/basal-like tumors are usually treated with some combination of surgery, radiation therapy and chemotherapy. These tumors cannot be treated with hormone therapies or trastuzumab (Herceptin®) because they are hormone receptor-negative and HER2/neu-negative.

### Methods of Assaying Nipple Aspirate Fluid (NAF)

In some embodiments, a method disclosed herein comprising determining the expression levels of a plurality of breast cancer biomarkers in a sample. In some embodiments, the sample is nipple aspirate fluid (NAF).

### Collection of NAF

NAF may be obtained by any suitable method. In some embodiments, NAF is collected by use of any absorbent paper. In some embodiments, the absorbent paper absorbs fluids. In some embodiments, the absorbent paper binds to proteins. In some embodiments, the absorbent paper does not bind to cells.

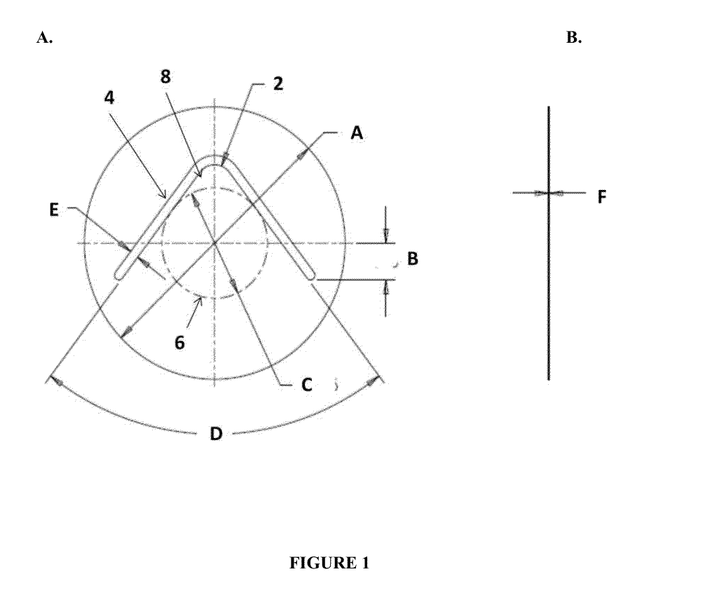

Absorbent papers 2 (which may also be called “membranes” herein) which may be used in the disclosed methods and may be any material that is suitable to collect epithelial cells and biomarkers such as, for example, proteins, carbohydrates, lipids, nucleic acids, RNA, DNA, etc. Absorbent papers 2 include those made of, for example, nitrocellulose, microcellulose, mixed cellulose ester, or any other appropriate material for nipple fluid sample collection. While FIG. 1A illustrates a circular absorbent paper, other shapes such as, for example, ovals, squares, triangles, other polygons, are also contemplated herein so long as the shape accommodates sample collection.

In some embodiments, the absorbent papers 2 does not cause papers cuts to the nipple and/or the areola. In some embodiments, the absorbent papers 2 is shaped to avoid paper cuts to the nipple and/or areola.

The absorbent papers 2 is formed by stamping the paper out of large paper stock with a metal mold. The absorbent papers 2 is big enough to cover or partially cover the nipple. In some embodiments, the absorbent papers 2 is big enough to cover the nipple. Therefore, an absorbent paper may be from about 1.0 inches to about 3.0 inches in diameter or length at its average dimension A across any size of the absorbent paper. An absorbent papers 2 may be, for example, about 1.0, about 1.1, about 1.15, about 1.2, about 1.25, about 1.3, about 1.35, about 1.4, about 1.45, about 1.5, about 1.55, about 1.6, about 1.65, about 1.7, about 1.75, about 1.8, about 1.85, about 1.9, about 1.95, about 2.0, about 2.1, about 2.15, about 2.2, about 2.25, about 2.3, about 2.35, about 2.4, about 2.45, about 2.5, about 2.55, about 2.6, about 2.65, about 2.7, about 2.75, about 2.8, about 2.85, about 2.9, about 2.95, or about 3.0 inches in diameter. FIG. 1A provides a non-limiting example of an absorbent papers 2 that is 1.85 inches in diameter A. In some embodiments, the absorbent papers 2 covers or partially covers the areola of a breast. In some embodiments, the absorbent papers 2 covers the areola of a breast. In some embodiments, the absorbent papers 2 partially covers the areola of a breast. In some embodiments, the absorbent paper covers a nipple and does not extend to the areola of a breast.

The thickness of the absorbent papers 2 may vary to allow for optimal sample collection and includes materials that are from about 0.01 inches to about 0.1 inches in thickness. For example, the absorbent papers 2 may be about 0.01, about 0.02, about 0.03, about 0.04, about 0.05, about 0.06, about 0.07, about 0.08 about 0.09, or about 0.1 inches thick. FIG. 1B provides a non-limiting example of an absorbent papers 2 that is 0.05 inches thick. One would understand that, while FIG. 1B illustrates the side view of an absorbent papers 2 that is 0.05 inches thick, the thickness can be varied as well.

The L-shaped element (which may also be identified as a “slit” herein) is optional and is useful if the absorbent papers 2 is placed in a pressure modifying device to lower the pressure and cause egress of fluid from the inside of the breast. In one embodiment, the absorbent papers 2 is sized such that it fits into a modified breast pump, and the dimensions are set accordingly. Modified breast pumps that may be optionally used in connection with an absorbent papers 2 are described herein.

The L-shaped element 4 in FIG. 1A is a cut out that remains after the die cut has stamped the paper. In one non-limiting example, the L-shaped element is 0.063 E inches across when cut out in the stamping process. In an absorbent papers 2 that is 1.85 inches A in diameter or length at its average dimension across any size of the device, the ends of the L-shaped element 4 are 0.25 inches B from the mid-line of the absorbent paper 2. The angle D of the L-shaped element 4 can be any angle from 10 degrees to 170 degrees D. In one non-limiting embodiment, the angle D is 75 degrees as illustrated in FIG. 1A. The inner circle 6 illustrated in FIG. 1A is approximately 0.75 inches C in diameter and was designed such that the L-shaped flap 8 moves properly when used in a breast pump device (e.g., a MASCT™ device described herein). The dash symbols designating the inner circle 6 illustrate guide lines in the figure. The slit 4 is shown by the incomplete triangle and is shaped as illustrated to form an incomplete circle. One would understand that the measurements described herein can be proportionally adjusted based upon the total size of the absorbent paper.

FIG. 1A represents the top view of one non-limiting example of an absorbent paper 2. The dash lines are not cut lines, but rather, are presented for ease of manufacturing to align the L-shaped element 4 such that the center of the absorbent papers 2 fits above the nipple area and so flap 8 sufficiently covers the nipple.

In another embodiment, the absorbent papers 2 is made of mixed cellulose ester and is formed in the shape and dimensions as illustrated in FIGS. 1A and 1B. In another embodiment, the absorbent paper or membrane is made of mixed cellulose ester and is formed in the shape and dimensions as illustrated in FIGS. 1A and 1B.

In some embodiments, a mammary fluid collection device is utilized to express and collect the NAF. In some embodiments, the mammary fluid collection device comprises a breast engaging portion or member coupled with a vacuum pump mechanism and may be fluidly connected with a solid phase sample collection medium comprising an absorbent paper (e.g., an absorbent paper that absorbs fluids, binds to proteins, and does not bind to cells).

In some embodiments, the mammary fluid collection device comprises a breast pump which is applied to a breast (e.g., a human breast) covering the nipple and used in conjunction with a nipple touch procedure as described in more detail below following use of the device (see also, Example 3).

In one embodiment, the nipple aspirate fluid is collected on an absorbent paper or membrane following massaging of breast tissue and suction with a MASCT™ device as described in Example 1 below. As used herein, a “MASCT™ device” refers to a device described in U.S. Pat. No. 6,287,521 by Quay et al. which is incorporated herein in its entirety. In one non-limiting example, a sample collection device for collecting a biological sample from a mammary organ of a patient may comprise a breast engaging member constructed of a non-porous material sized and dimensioned to receive at least a nipple portion of a breast of said patient and form a suction seal therewith; a solid phase sample collection medium in fluid connection with said breast engaging member for receiving a sample of expressed breast fluid; and a vacuum pump means in gaseous connection with said breast engaging member for generating negative pressure through the breast engaging member to facilitate breast fluid expression, wherein said solid phase sample collection medium is selected from the group consisting of microscopic glass slides, capillary tubes, collection tubes, columns, micro-columns, wells, plates, membranes, filters, resins, inorganic matrices, beads, particulate chromatographic media, plastic microparticles, latex particles, coated tubes, coated templates, coated beads, coated matrices, or a combination thereof. The sample collection device may include removable coupling means for removably coupling said sample collection housing with said breast engaging member. In some instances, the solid phase sample collection medium is supported by a support member integrally or removably mounted within said sample collection housing in fluid connection with said breast engaging member. The support member may be disc-shaped and is interposed between said breast engaging member and said sample collection housing. Further, the support member may have upper and lower retaining rings and supports a sheet of absorbent or adsorbent material. Support member supports may be a solid phase sample collection template including, but not limited to, capillary tubes, coated tubes, columns, micro-columns, plates, wells and microscopic slides, or a combination thereof. Support members define a fluid-retaining well and include at least one air channel to allow negative pressure to pass through the air channel to and from said breast engaging member. The solid phase sample collection medium may be a particulate medium contained within a cartridge removably mounted within said sample collection housing and having a first end of said cartridge in fluid connection with said breast engaging member where the first end of said cartridge is covered by a porous barrier material.

In some embodiments, the nipple touch procedure is administered by applying an absorbent paper as described herein to each nipple.

During or after administration of the device to produce mammary fluid, a biological sample is collected from the expressed mammary fluid using the absorbent paper, which sample may contain one or more of whole mammary fluid, whole cells, cell fragments, cell membranes, selected liquid, cellular or other solid fractions of the mammary fluid, as well as proteins, glycoproteins, peptides, nucleotides (including DNA, RNA, etc.) and other like biochemical and molecular constituents of the mammary fluid.

Following contact with the NAF, the absorbent paper is washed and the effluent is collected and assessed for number of cells. Where the sample is acellular, in some embodiments the patient is identified as at low risk for breast cancer. Where the sample comprises one cell, in some embodiments the patient is identified as at low risk for breast cancer, and optionally the cells are assayed for biomarker expression. Where the sample comprises 2 or more cells, in some embodiments the patient is identified as at risk for breast cancer and the cells are assayed for biomarker expression.

### Breast Cancer Biomarkers

Breast ducts contain two types of epithelial cells, inner luminal cells and outer basal/myoepithelial cells. In some embodiments, biomarker expression (e.g., by immunohistochemical staining) is used to distinguish between luminal and basal breast cancers. In some embodiments, biomarker expression (e.g., by immunohistochemical staining) is used to distinguish between hyperplasia of the usual type and atypical hyperplasia.

Tumor protein p63 (or, transformation-related protein 63) is a member of the p53 family of nuclear transcription factors. Tumor protein p63 is encoded by the TP63 gene. The presence of p63 characterizes the basal epithelial layer. In some embodiments, the presence of p63 in a nipple aspirate fluid (NAF) cell indicates that a breast cancer is basal-like breast cancer.

The presence of cytokeratin (CK) 5 and CK14 characterizes the basal epithelial layer. In some embodiments, the presence of CK5 and CK14 in a nipple aspirate fluid (NAF) cell indicates that a breast cancer is basal-like breast cancer. Further, the presence of CK5 and CK14 characterizes progenitor and myoepithelial cells. In some embodiments, the presence of CK5 and CK14 in a nipple aspirate fluid (NAF) cell indicates that the cell is a myoepithelial cell or a progenitor cell.

The presence of CK7 and CK18 characterizes the luminal epithelial layer. In some embodiments, the presence of CK7 and CK18 in a nipple aspirate fluid (NAF) cell indicates that a breast cancer is luminal breast cancer.

Usual ductal hyperplasia displays a luminal staining pattern with expression of both CK5/14 and CK7/18. Residual p63 is observed in the nuclei of the myoepithelium. In some embodiments, the presence of CK5, CK14, CK7, and CK18 indicates that a hyperplasia is usual ductal hyperplasia.

Atypical ductal hyperplasia or ductal carcinoma in situ display the differentiated glandular immunophenotype (CK7/CK18 positive), but are CK5/14-negative except for the myoepithelium. In some embodiments, the presence of CK7/CK18 and the absence of CK5/14 indicate that a hyperplasia is atypical ductal hyperplasia.

Invasive breast lesions are identified by a reduction in the number of or absence of myoepithelial cells (CK5/14 and/or p63) and the presence of glandular epithelial cells (CK7/18). Myoepithelial cells are ‘contractile’—they function to forcibly express the contents of a gland. In the breast, the myoepithelial cells are located above the basal layer and just below the top layer of secretory cells at the duct wall. The presence of reduced or under-stress myoepithelial cells in the context of a suspected breast cancer can be of concern, and may indicate a transition to infiltrating and possibly invasive status. Primary breast carcinomas show an increase in the number of luminal (duct-wall) cells and a decrease in the number of myoepithelial cells. As a breast cancer evolves from in-situ, to infiltrating, and finally to invasive, the relative number of myoepithelial cells decreases. If the finding is for larger than normal numbers of luminal cells, it suggests that myoepithelial cells are diminishing in number, and there is cause for concern. In some embodiments, the absence of or a reduction in the number of myoepithelial cells and the presence of glandular epithelial cells indicates that the lesion is invasive.

In some embodiments, biomarker expression is determined by immunohistochemistry. In some embodiments, the immunohistochemistry method is a direct method. In some embodiments, a cell isolated from NAF is contacted with a labeled antibody binds to the target antigen. Any suitable label may be used with a method disclosed herein. In some embodiments, the label is a dye (or, stain). In some embodiments, a different dye is used for each antibody. In some embodiments, the same dye is used for antibodies that bind to biomarkers present in the same cells type. For example, a first dye is used for antibodies that bind to biomarkers present in luminal breast cancer cells (CK7/18) and a second dye is used for antibodies that bind to biomarkers present in basal breast cancer cells (CK5/14 and p63).

In some embodiments, the immunohistochemistry method is an indirect method. In some embodiments, a cell isolated from NAF is contacted with an unlabeled primary antibody and binds to the target antigen and a labeled secondary antibody binds to the primary antibody. In some embodiments, the primary antibody binds to a biomarker (e.g., CK5, CK7, CK14, CK18, or p63). In some embodiments, horseradish peroxidase (HRP) secondary antibodies bind to antibodies that bind to CK5/14 and p63. In some embodiments, alkaline phosphatase (AP) secondary antibodies bind to antibodies that bind to CK7/18. In some embodiments, a secondary antibody is raised to react with a primary antibody based on the species origin of the primary antibody, e.g., if the primary antibody is a mouse antibody then the secondary antibody would be, for example, a rabbit anti-mouse antibody. In a preferred embodiment, a conjugated goat anti-mouse poly-alkaline phosphatase (ALP) and a conjugated goat anti-rabbit poly-horseradish peroxidase (HRP) are used as secondary antibodies and react with both heavy and light chains on mouse and rabbit IgG.

In some embodiments, a chromogen (e.g., 3,3′ diaminobenzidine (DAB)) binds to the HRP and produces a chromogenic reaction product. Where the chromogen is DAB, the chromogen reaction product is brown. When the chromogen is Bajoran Purple, the chromogen reaction product is lavender-purple. I

In some embodiments, a chromogen (e.g., Fast Red (FR)) binds to the AP and produces a chromogenic reaction product. Where the chromogen is FR, the chromogen reaction product is red or pink. In some embodiments, a cell isolated from NAF is contacted with a peroxide block before contact with the primary antibody. Where the chromogen is Ferangi Blue, the chromogen reaction product is a bright royal blue.

In some embodiments, the cells are counterstained. In some embodiments, the cells are counterstained with hematoxylin, Nuclear Fast Red, Methyl Green, or Methyl Blue.

Antibodies that bind to CK5, CK7, CK14, CK18, or p63 to be used in the presently described methods are commercially available from, for example, Genetex, Beckman Coulter, Imgenex, Spring Bioscience, BD Biosciences, Raybiotech, Inc., Biorbyt, Abnova Corp., Proteintech Group, Fitzgerald Industries, Inc., Biocare Medical, Santa Cruz Biotechnology, Inc., and LifeSpan Biosciences. Antibodies may also be made according to conventional antibody techniques.

### Digital Processing Device

In some embodiments, the methods, systems, and software described herein include a digital processing device, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

### Non-Transitory Computer Readable Storage Medium

In some embodiments, the methods, systems, and software disclosed herein include one or more computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the methods, systems, and software disclosed herein include at least one computer program, or use of the same. A computer program includes a sequence of instructions, executable in the digital processing device's CPU, written to perform a specified task. In light of the disclosure provided herein, those of skill in the art will recognize that a computer program may be written in various versions of various languages. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. A web application for providing a career development network for artists that allows artists to upload information and media files, in some embodiments, includes a media player element. In various further embodiments, a media player element utilizes one or more of many suitable multimedia technologies including, by way of non-limiting examples, Adobe® Flash®, HTML 5, Apple® QuickTime®, Microsoft Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

The methods, systems, and software disclosed herein include, in various embodiments, software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the methods, systems, and software disclosed herein include one or more databases, or use of the same. In view of the disclosure provided herein, those of skill in the art will recognize that many databases are suitable for storage and retrieval of metagenomic information (including metagenomic profiles), metatranscriptome information (including metatranscriptome profiles), and multiplex profiles. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local storage devices.

### EXAMPLES

### Example 1

### Immunohistochemical Analysis of Biomarkers

Following collection of a NAF sample on an absorbent paper, the absorbent paper is washed using any suitable buffered wash solution (e.g., phosphate buffered saline). The effluent is collected in a modified cytology vial and centrifuged. Cells are isolated from the effluent and transferred to the central region of a clean glass microscopic slide, and a cover slip is applied. The slide is allowed to air dry and then is fixed, for example in absolute alcohol.

Monoclonal antibodies CK5, CK14, p63 and rabbit monoclonal antibodies CK7 and CK18 are multiplexed with a single antibody diluent and applied to the microscopy slide. A biotin-free multistain detection reagent composed of a cocktail of goat-anti-mouse-HRP and goat anti-rabbit-AP is then applied. DAB and Fast Red chromogens are applied sequentially. Cells are counterstained with hematoxylin.

| Antibody | Chromogen | Cell Type |
|---|---|---|
| CK5 | DAB | Progenitor cells |
| | Brown | Myoepithelial/luminal |
| | | Basal phenotype |
| CK14 | DAB | Progenitor cells |
| | Brown | Myoepithelial/luminal |
| | | Basal phenotype |
| P63 | DAB | Basal Myoepithelium |
| | Brown | Basal phenotype |
| CK7 | FR | Normal breast cells |
| | Red | Glandular epithelium |
| | | Luminal epithelium |
| CK18 | FR | Normal breast cells |
| | Red | Glandular epithelium |
| | | Luminal epithelium |

### Example 2

### Assessment of Nipple Aspirate Fluid Using the ADH5 Assay

Following collection of a NAF sample on an absorbent paper, the absorbent paper is washed using any suitable buffered wash solution (e.g., phosphate buffered saline). The effluent is collected in a modified cytology vial and centrifuged. Cells are isolated from the effluent and transferred to the central region of a clean glass microscopic slide, and a cover slip is applied. The slide is allowed to air dry and then is fixed, for example in absolute alcohol.

### Pretreatment

The cells are contacted with a peroxide block—Biocare's Peroxidazed 1.

Next, perform heat retrieval pretreatment. Preheat Diva solution to 95° C. for 30 minutes in Biocare's Decloaking Chamber. Then, place slides into the preheated solution and retrieve under pressure at 95° C. for 40 minutes. Alternatively, steam tissue sections for 45-60 minutes or use a water bath at 95° C. for 40 minutes. Allow solution to cool for 20 minutes then wash in distilled water.

Apply protein block—Incubate for 10-15 minutes at RT with Biocare's Background Sniper.

Incubate the slide with the primary antibodies (i.e., antibodies to CKS, CK14, CK7, CK18, and p63) for 30-60 minutes at room temperature.

Incubate slide for 30 minutes at RT using Biocare's MACH 2 Double Stain 2.

Incubate for 5 minutes at RT when using Biocare's Betazoid DAB.

Incubate for 10-20 minutes at RT with Biocare's Vulcan Fast Red. Rinse in deionized water.

Rinse with deionized water. Incubate for 30-60 seconds with Hematoxylin. Rinse with deionized water. Apply Tacha's Bluing solution for 1 minute.

Visualize cells with a light microscope.

### Example 3

### Assessment of Nipple Aspirate Fluid

This trial was a single-center study involving three (3) healthy, non-pregnant, non-lactating female subjects. Subjects were enrolled in the order of appearance at the clinic.

The primary trial objective was to determine the percentage of women from age 30 to 65 that produces ductal fluid, as determined by the presence of protein on the nitrocellulose filter when using the MASCT™ device.

A secondary objective was to evaluate the nipple aspirate fluid cytologically for the presence and type of cells (if any).

### Abbreviations

Abbreviations used herein include, for example, MAF: Mammary Aspiration Fluid; MASCT™: Mammary Aspiration Specimen Cytology Test; NA: Not Available; ND: Not Done; NR: Not Recorded; and NAF: Nipple Aspirate Fluid.

### Methodology:

Briefly, a tared nitrocellulose filter was used to collect ductal fluid by just touching it to each nipple (one for each breast). Next, mammary fluid samples were aspirated using the MASCT™ device with a tared sample collection unit. Both sets of nitrocellulose filters were tested for protein using a staining technique described below. Cells collected from washing the filters containing nipple aspirate fluid specimens underwent cytological examination.

### Assessment:

The primary endpoint of the trial was the percentage of women completing the trial that produce ductal fluid, as determined by the presence of protein on the nitrocellulose filter when using the MASCT™ device.

The secondary endpoint was the presence of cells in the nipple aspirate fluid as determined by cytologic evaluation.

### Results:

With regard to the protein testing done of the filters obtained from these 3 subjects, none on the nipple wash filter samples showed the presence of protein. All filters from the MASCT™ device showed that protein was detected on the device filter.

### Overall Study Design and Plan

The MASCT™ device had been previously cleared for marketing via the 510(k) regulatory pathway. This clinical study was designed to test modifications to the MASCT™ device that were made to enhance efficacy and usability and the ability to detect protein in nipple aspirate fluid from women, including those previously thought to be non-secreters. The clinical utility of nipple aspirate fluid for helping in breast health management has been hampered over the last 50 years by the current methodology of collecting and measuring the presence of fluid. In fact, with current technology up to 50% of all women are non-secretors, that is, they are judged to not produce NAF.

This was a single-center study involving the enrollment of up to 50 healthy non-pregnant, non-lactating female subjects. Subjects were enrolled in the order of appearance at the clinic.

Prior to entering the study, the investigator or designated assistant explained to each subject, the nature of the study, its purpose, procedures, expected duration, available alternatives, and the benefits and risks involved in study participation. Each subject was given a consent document and had the opportunity to ask questions; and was informed of her right to withdraw from the study at any time without prejudice. After this explanation, and before any study-specific procedures are performed, the subject voluntarily signed and dated an informed consent statement. Prior to participation in the study, each subject received a copy of the signed and dated written informed consent form and any other written information.

### Inclusion/Exclusion Criteria Review and Pregnancy Evaluation

When necessary, each patient underwent a urine pregnancy test prior to further participation in the study. A positive pregnancy test would exclude the subject from participation. All inclusion and exclusion criteria were reviewed to ensure subject eligibility. After eligibility was established, a unique subject identification number was assigned.

### Demographics and Medical History

The following demographic and medical history was obtained from each subject: age and ethnic origin; family medical history, especially mother and sisters; personal medical history, including breast cancer, benign breast conditions, and reproductive diseases (for example, ovarian or endometrial tumors); concomitant medications; age of menarche; age at first pregnancy; age at first live birth; age of menopause; and height and weight.

### Breast Preparation

Subjects were placed in a recumbent position. The nipple and peri-aureolar areas of both breasts were cleansed with alcohol to remove excess skin oils, cosmetics or epithelial debris. After the alcohol evaporated, a warm moist compress was placed on both breasts for 10 to 15 minutes. The compresses were removed and the subjects were placed in a seated position. Alcohol was used to wipe the nipple area to remove any ductal plugs that were present.

### Nipple Touch Procedure

All persons handling filter materials shall wear gloves and a protective mask to minimize the risk of filter contamination.

### Device Cleaning Procedure

Prior to each subject use, the MASCT™ device was thoroughly cleaned with an antimicrobial solution such as CIDEX®. The device was not exposed to extreme temperatures or autoclaved. The device was inspected periodically for deterioration of the materials of the device or failure to induce negative pressure. If either condition was observed, the unit was replaced.

### MASCT™ and Nipple Touch Procedures

a. Label one filter disk assembly ‘left’ and a second filter disk assembly ‘right’.
b. Weigh and record the weights of each assembly taking care to not touch the filter with a bare hand, and using forceps as needed.
c. Insert one assembly into the breast pump device.
d. Instruct the subject to perform manual self-breast massage beginning with the chest wall and moving gradually to the nipple-aureolar complex for approximately one minute.
e. The subject will then compress her breast with both hands while the breast pump device is actuated for 60-90 seconds by the physician or nurse practitioner.
f. Remove the filter disk assembly and weigh the MASCT™ filter disks for fluid collection.
g. Using an antimicrobial solution such as CIDEX®, wipe the surfaces of the sample collection funnel and rinse out the collection vial.
h. Repeat steps c, d, e, f and g for the second breast.
i. Store the filter disks in a refrigerator for subsequent evaluation of protein and/or cytology evaluation. The packaging for each filter disk assembly shall be appropriately labeled with subject ID number and date of collection.

Variations to this collection procedure may be conducted, but at no time will the number of massage/collection procedures exceed that allowed in the approved labeling for the MASCT™ device.

### Subject Observation

Subjects will remain in the clinic and be observed for adverse device effects for 30 minutes following aspiration. If there are no evolving or unresolved adverse effects at that time, the subjects will be released. At the investigator's discretion, subjects experiencing any adverse effects at the end of this observation period will remain at the study site until either the effect resolves or follow-up arrangements have been made.

### Conduct of the Study

All subjects in this study were studied according to the protocol described herein.

Three (3) subjects were enrolled and the disposition of patients is provided in the table below.

SUBJECT DISPOSITION BY SUBJECT

| Subject number | Enrolled | Completed Study |
|---|---|---|
| B01 | Yes | Yes |
| B02 | Yes | Yes |
| B03 | Yes | Yes |

The following table contains individual patient data listings for demographics. There were no deaths or serious adverse events.

SUBJECT DEMOGRAPHICS

| | | | Active |
|---|---|---|---|
| Age | | Mean | 50 |
| | | Std. Dev. | 11.79 |
| | | Median | 53 |
| | | Range | 37-60 |
| Weight (kg) | | Mean | 135.5 |
| | | Std. Dev. | 20.5 |
| | | Median | 135.5 |
| | | Range | 115-156 |
| Height (cm) | | Mean | 64.5 |
| | | Std. Dev. | 3.04 |
| | | Median | 66 |
| | | Range | 61-66.5 |
| BMI | | Mean | 23.03 |
| | | Std. Dev. | 4.1 |
| | | Median | 25.2 |
| | | Range | 18.3-25.6 |
| | | N | % |
| Gender | Female | 3 | 100 |
| Ethnicity | Caucasian | 3 | 100 |

### Discussion and Overall Conclusions

The following results were obtained upon testing of the individual filters used in this study.

### Nipple Wash Results

None of the control nipple wash filter samples demonstrated any presence of protein.

Breast Pump Results

| Subject | Breast | Concentration of protein |
|---|---|---|
| Subject #1 | Left Breast | 30 ng of protein |
| Subject #1 | Right Breast | 868 pg of protein |
| Subject #2 | Left Breast | 580 pg of protein |
| Subject #2 | Right Breast | 71 pg of protein |
| Subject #3 | Left Breast | 7.1 ng of protein |
| Subject #3 | Right Breast | 17.6 ng of protein |

Based on these results, it was determined that the MASCT™ device obtained nipple aspirate fluid as evidenced by the presence of protein.

### Deaths or Serious Adverse Events

There were no deaths or serious adverse events.

### Subsequent Data

In a study of 31 women aged 18-65, the method of this disclosure was used to analyze for protein and it was detected in all 31 women (one woman had protein detected in only one breast) giving a clinical utility of 97%. This improvement over the prior art makes this test useful for identifying women with early, precancerous changes when cancer prevention measures can be instituted.

### A Summary of the Efficacy Evaluation

The primary objective of the study was achieved as determined by the presence of protein on all nitrocellulose filters obtained when using the MASCT™ device. The secondary objective of evaluating nipple aspirated fluid cytologically for the presence and type of cells was accomplished. All samples were successfully analyzed for cellular material.

While preferred embodiments have been shown and described herein, it will be obvious to those skilled in the art that such embodiments are provided by way of example only. Numerous variations, changes, and substitutions will now occur to those skilled in the art without departing from the embodiments. It should be understood that various alternatives to the embodiments described herein may be employed. It is intended that the following claims define the scope of the embodiments and that methods and structures within the scope of these claims and their equivalents be covered thereby.

1.-22. (canceled)

23. A method of classifying a breast cancer, comprising:
a) contacting a cell of a nipple aspirate fluid sample absorbed onto an absorbent paper with antibodies that bind to CK5, CK14, CK7, CK18, and p63, wherein the absorbent paper is sized to cover a nipple;
b) detecting binding of one or more of the antibodies to said cell; and
c) classifying the cancer based upon the binding pattern of the primary antibodies;
wherein the cell derived from the nipple aspirate fluid (NAF) sample is not a tissue.

24. The method of claim 23, wherein the absorbent paper is from about 1.0 to about 3.0 inches in diameter and from about 0.01 to about 0.1 inches thick.

25. The method of claim 23, wherein detecting binding of the one or more antibodies further comprises staining the cells with a stain selected from among horseradish peroxidase, alkaline phosphatase, diaminobenzidine, Fast Red, hematoxylin, eosin and a combination thereof.

26. The method of claim 23, further comprising washing the absorbent paper and collecting the effluent.

27. The method of claim 23, wherein the absorbent paper comprises microcellulose, mixed cellulose ester, or nitrocellulose.

28. The method of claim 23, wherein the absorbent paper is a device as illustrated in FIG. 1.

29. The method of claim 23, further comprising classifying the breast cancer as basal-like if an anti-CK5 antibody, an anti-CK14 antibody, and optionally an anti-p63, primary antibody binds to the cell.

30. The method of claim 23, further comprising classifying the breast cancer as luminal if (i) an anti-CK7 antibody and an anti-CK18 primary antibody bind to the cell, and (ii) an anti-CK5 antibody, and anti-CK14 antibody, and an anti-p63 antibody do not bind to the cell.

31. The method of claim 23, further comprising classifying the breast cancer as usual ductal hyperplasia if an anti-CK5 antibody, an anti-CK14 antibody, an anti-CK7 antibody, an anti-CK18 antibody, and an anti-p63 primary antibody bind to the cell.

32. The method of claim 23, further comprising classifying the cancer as atypical ductal hyperplasia if (i) the anti-CK7 antibody and anti-CK18 antibody, and optionally the anti-p63 antibody, bind to the cell, and (ii) the anti-CK5 antibody and anti-CK14 antibody do not bind to the cell.

33. The method of claim 23, further comprising classifying the cancer as invasive if (i) the sample comprises more than one cell and (i) the ratio of cells binding the anti-CK5 antibody, anti-CK14 antibody, and anti-p63 antibody to cells binding the anti-CK7 antibody and anti-CK18 antibody is less than or equal to an invasive control.

34. The method of claim 23, further comprising classifying the cancer as non-invasive if: (i) the sample comprises more than one cell, and (ii) the ratio of cells binding the anti-CK5 antibody, anti-CK14 antibody, and anti-p63 antibody to cells binding the anti-CK7 antibody and anti-CK18 antibody is greater than or equal to a non-invasive control.

35. (canceled)

36. A composition comprising (a) at least one cell derived from nipple aspirate fluid absorbed onto an absorbent paper comprising microcellulose, mixed cellulose ester, or nitrocellulose for absorbing a nipple aspirate fluid sample, wherein the absorbent paper is sized to cover a nipple; and (b) antibodies that bind to an antigens on a cell in the nipple aspirate fluid sample, wherein the antigens are selected from: CK5, CK14, CK7, CK18, p63, CK7 and CK18.

37. A system for classifying a breast cancer, comprising:
a) an absorbent paper comprising microcellulose, mixed cellulose ester, or nitrocellulose for absorbing a nipple aspirate fluid sample, wherein the absorbent paper is sized to cover a nipple; and
b) antibodies that bind to an antigens on a cell in the nipple aspirate fluid sample, wherein the antigens are selected from: CK5, CK14, CK7, CK18, p63, CK7 and CK18.

38. The system of claim 37, further comprising a light microscope or an automated system for visualizing the antibodies bound to a cell in the nipple aspirate fluid sample.

39. The system of claim 37, wherein the absorbent paper is from about 1.0 to about 3.0 inches in diameter and from about 0.01 to about 0.1 inches thick.

40. The system of claim 37, further comprising means for visualizing the antibodies bound to the cell in the nipple aspirate fluid sample.

41. The system of claim 40, wherein the means for visualizing the antibodies bound to the cell in the nipple aspirate fluid sample is one or more stains.

42. The system of claim 41, wherein the stain is selected from horseradish peroxidase, alkaline phosphatase, diaminobenzidine, Fast Red, hematoxylin, eosin or a combination thereof.

43. (canceled)

44. The system of claim 37, further comprising: an optionally networked computer processing device configured to perform executable instructions; and a computer program, the computer program comprising a software module executed by the computer processing device to apply a model or algorithm for analyzing said cells.

45.-52. (canceled)
